(19) United States
(12) Patent Application Publication
(10) Pub. No.: US 2026/0158033 A1
(43) Pub. Date: Jun. 11, 2026

(54) VECABRUTINIB FOR THE TREATMENT OF GRAFT-VERSUS-HOST DISEASE

(71) Applicants: Viracta Therapeutics, Inc., Cardiff, CA (US); H. Lee Moffitt Cancer Center and Research Institute, Inc., Tampa, FL (US)

(72) Inventors: Judith A. FOX, San Francisco, CA (US); Pietro TAVERNA, Watertown, MA (US); Kamira MAHARAJ, Tampa, FL (US); Javier PINILLA, Tampa, FL (US); Eva SAHAKIAN, Tampa, FL (US)

(21) Appl. No.: 18/706,905

(22) PCT Filed: Nov. 2, 2022
(86) PCT No.: PCT/US2022/079155

§ 371 Date: May 2, 2024

Related U.S. Application Data
(60) Provisional application No. 63/275,171, filed on Nov. 3, 2021.

(51) Int. Cl. A61K 31/506; A61P 37/06

(52) U.S. Cl. CPC A61K 31/506; A61P 37/06

(57) ABSTRACT

The present disclosure relates to methods of treating or preventing chronic graft-versus-host disease with a BTK inhibitor (e.g., vecabrutinib).

### RELATED APPLICATIONS

This application claims priority to U.S. Provisional Application No. 63/275,171, filed Nov. 3, 2021, the entire contents of which are incorporated herein by reference.

### FIELD OF DISCLOSURE

The present disclosure relates to methods of treating chronic graft-versus-host disease with a BTK inhibitor (e.g., vecabrutinib).

### BACKGROUND

Chronic graft-versus-host disease (cGVHD) can manifest as a complication in patients following allogeneic hematopoietic stem cell transplant resulting in morbidity and mortality. cGVHD is estimated to develop in approximately 40% of transplant recipients, is estimated to effect 14,000 patients in the US and can last for years. Chronic GVHD typically manifests across multiple organ systems, with the skin and mucosa being commonly involved and is characterized by the development of fibrotic tissue. While the pathophysiological understanding of cGVHD is emerging, there has been little meaningful development of therapies for patients with cGVHD. Effective treatment strategies for cGVHD are currently lacking.

Ibrutinib, an irreversible BTK inhibitor with activity against several other Tec-family kinases, has been clinically developed for cGVHD treatment due to regulation of pathogenetic B cells and T-cell subsets. The side effects of ibrutinib are significant with 38% of patients discontinuing due to an adverse event and 310% of patients dose reducing.

In contrast to ibrutinib and other BTK inhibitors, vecabrutinib (i.e., (3R,3′R,4'S)-1′-(6-amino-5-fluoropyrimidin-4-yl)-3-((3-chloro-5-(trifluoromethyl) phenyl)amino)-2-oxo-[1,3′-bipiperidine]-4′-carboxamide) is a potent, reversible, non-covalent BTK inhibitor. Vecabrutinib has a distinct kinase domain interaction, which could result in differentiated safety and activity profiles compared to ibrutinib. Vecabrutinib has the capacity to overcome the C481S mutation that mediates resistance to ibrutinib. Vecabrutinib also demonstrates activity against ITK, which is expressed in T cells. In this study, we investigated the activity and immune modulation of vecabrutinib treatment in a murine model of sclerodermatous cGVHD.

### SUMMARY

The present disclosure relates to the use of a BTK inhibitor (e.g., vecabrutinib) for the treatment of graft-versus-host disease (GVHD).

In some aspects, the present disclosure provides a method of treating or preventing a disease or disorder in a subject in need thereof, comprising administering to the subject a therapeutically effective amount of vecabrutinib, or a pharmaceutically acceptable salt thereof, or pharmaceutical composition disclosed herein.

In some aspects, the present disclosure provides vecabrutinib, or a pharmaceutically acceptable salt thereof, or pharmaceutical composition disclosed herein for use in treating or preventing a disease or disorder.

In some aspects, the present disclosure provides a use of vecabrutinib, or a pharmaceutically acceptable salt thereof, in the manufacture of a medicament for treating or preventing a disease or disorder.

In some embodiments, treating is reducing the severity of the disease or disorder occurrence in a subject.

In some embodiments, vecabrutinib is administered after cell transplantation in the subject.

In some embodiments, the disease or disorder is graft-versus-host disease.

In some embodiments, the graft-versus-host disease is chronic graft-versus-host disease (cGVHD).

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the peptides disclosed herein, the chemical structures will control.

Other features and advantages of the disclosure will be apparent from the following detailed description and claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts the clinical score in treated mice.

FIG. 2 depicts the pathogenic B-cell characteristics of mice treated with vehicle, vecabrutinib, and ibrutinib at Day 40 and Day 76.

FIG. 3A and FIG. 3B depict the T-cells phenotype and function of mice treated with vehicle, vecabrutinib, and ibrutinib at Day 40 and Day 76.

FIG. 4 depicts the cytokine-secreting B-cells and T-cells characteristics of mice treated with vehicle, vecabrutinib, and ibrutinib at Day 40 and Day 76.

FIG. 5 depicts the circulating immunoglobulins (i.e., IgA, IgG1, IgG2b, and IgM) of mice treated with vehicle, vecabrutinib, and ibrutinib at Day 40 and Day 76.

### DETAILED DESCRIPTION

In some aspects, the present disclosure provides a method of treating or preventing a disease or disorder in a subject in need thereof, comprising administering to the subject a therapeutically effective amount of a BTK inhibitor, or a pharmaceutically acceptable salt thereof, or pharmaceutical composition disclosed herein.

In some aspects, the present disclosure provides a method of treating a disease or disorder in a subject in need thereof, comprising administering to the subject a therapeutically effective amount of a BTK inhibitor, or a pharmaceutically acceptable salt thereof, or pharmaceutical composition disclosed herein.

In some aspects, the present disclosure provides a method of treating or preventing a disease or disorder in a subject in need thereof, comprising administering to the subject a therapeutically effective amount of vecabrutinib, or a pharmaceutically acceptable salt thereof, or pharmaceutical composition disclosed herein.

In some aspects, the present disclosure provides a method of treating a disease or disorder in a subject in need thereof, comprising administering to the subject a therapeutically effective amount of vecabrutinib, or a pharmaceutically acceptable salt thereof, or pharmaceutical composition disclosed herein.

In some aspects, the present disclosure provides a method of treating or preventing graft-versus-host disease in a subject in need thereof, comprising administering to the subject a therapeutically effective amount of vecabrutinib, or a pharmaceutically acceptable salt thereof, or pharmaceutical composition disclosed herein.

In some aspects, the present disclosure provides a method of treating graft-versus-host disease in a subject in need thereof, comprising administering to the subject a therapeutically effective amount of vecabrutinib, or a pharmaceutically acceptable salt thereof, or pharmaceutical composition disclosed herein.

In some aspects, the present disclosure provides a method of treating or preventing chronic graft-versus-host disease (cGVHD) in a subject in need thereof, comprising administering to the subject a therapeutically effective amount of vecabrutinib, or a pharmaceutically acceptable salt thereof, or pharmaceutical composition disclosed herein.

In some aspects, the present disclosure provides a method of treating chronic graft-versus-host disease (cGVHD) in a subject in need thereof, comprising administering to the subject a therapeutically effective amount of vecabrutinib, or a pharmaceutically acceptable salt thereof, or pharmaceutical composition disclosed herein.

In some aspects, the present disclosure provides a BTK inhibitor, or a pharmaceutically acceptable salt thereof, or pharmaceutical composition disclosed herein for use in treating or preventing a disease or disorder.

In some aspects, the present disclosure provides a BTK inhibitor, or a pharmaceutically acceptable salt thereof, or pharmaceutical composition disclosed herein for use in treating a disease or disorder.

In some aspects, the present disclosure provides vecabrutinib, or a pharmaceutically acceptable salt thereof, or pharmaceutical composition disclosed herein for use in treating or preventing a disease or disorder.

In some aspects, the present disclosure provides vecabrutinib, or a pharmaceutically acceptable salt thereof, or pharmaceutical composition disclosed herein for use in treating a disease or disorder.

In some aspects, the present disclosure provides vecabrutinib, or a pharmaceutically acceptable salt thereof, or pharmaceutical composition disclosed herein for use in treating or preventing graft-versus-host disease.

In some aspects, the present disclosure provides vecabrutinib, or a pharmaceutically acceptable salt thereof, or pharmaceutical composition disclosed herein for use in treating graft-versus-host disease.

In some aspects, the present disclosure provides vecabrutinib, or a pharmaceutically acceptable salt thereof, or pharmaceutical composition disclosed herein for use in treating or preventing chronic graft-versus-host disease (cGVHD).

In some aspects, the present disclosure provides vecabrutinib, or a pharmaceutically acceptable salt thereof, or pharmaceutical composition disclosed herein for use in treating chronic graft-versus-host disease (cGVHD).

In some aspects, the present disclosure provides a use of a BTK inhibitor, or a pharmaceutically acceptable salt thereof, in the manufacture of a medicament for treating or preventing a disease or disorder.

In some aspects, the present disclosure provides a use of a BTK inhibitor, or a pharmaceutically acceptable salt thereof, in the manufacture of a medicament for treating a disease or disorder.

In some aspects, the present disclosure provides a use of vecabrutinib, or a pharmaceutically acceptable salt thereof, in the manufacture of a medicament for treating or preventing a disease or disorder.

In some aspects, the present disclosure provides a use of vecabrutinib, or a pharmaceutically acceptable salt thereof, in the manufacture of a medicament for treating a disease or disorder.

In some aspects, the present disclosure provides a use of vecabrutinib, or a pharmaceutically acceptable salt thereof, in the manufacture of a medicament for treating or preventing graft-versus-host disease.

In some aspects, the present disclosure provides a use of vecabrutinib, or a pharmaceutically acceptable salt thereof, in the manufacture of a medicament for treating graft-versus-host disease.

In some aspects, the present disclosure provides a use of vecabrutinib, or a pharmaceutically acceptable salt thereof, in the manufacture of a medicament for treating or preventing chronic graft-versus-host disease (cGVHD).

In some aspects, the present disclosure provides a use of vecabrutinib, or a pharmaceutically acceptable salt thereof, in the manufacture of a medicament for treating chronic graft-versus-host disease (cGVHD).

In some embodiments, treating is reducing the severity of the disease or disorder occurrence in a subject.

Treating the conditions listed herein can result in preventing the occurrence of the conditions described herein, including chronic graft versus host disease (cGVHD) or reducing the severity of cGVHD. A reduction in symptoms may also be referred to as “regression”. Preferably, after treatment, severity is reduced by 5% or greater relative to prior to treatment; more preferably, severity is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Severity may be measured by any reproducible means of measurement. The severity may be measured as a diameter of the area of interest or according to various physician scales.

In some embodiments, the BTK inhibitor is vecabrutinib.

In some embodiments, the disease or disorder is graft-versus-host disease (GVHD).

In some embodiments, the graft-versus-host disease is acute GVHD.

In some embodiments, the graft-versus-host disease is chronic graft-versus-host disease (cGVHD).

In some embodiments, the GVHD is solid organ transplant graft-versus-host disease (SOT-GVHD).

In some embodiments, the cGVHD is liver cGVHD. In some embodiments, the cGVHD is kidney cGVHD. In some embodiments, the cGVHD is esophageal cGVHD. In some embodiments, the cGVHD is stomach cGVHD. In some embodiments, the GVHD is sclerodermatous GVHD. In some embodiments, the GVHD is steroid resistant GVHD. In some embodiments, the GVHD is cyclosporin-resistant GVHD. In some embodiments, the GVHD is refractory GVHD. In some embodiments, the GVHD is oral GVHD. In some embodiments, the oral GVHD is reticular oral GVHD. In some embodiments, the oral GVHD is erosive oral GVHD. In some embodiments, the oral GVHD is ulcerative oral GVHD. In some embodiments, the oral GVHD is GVHD of the oral cavity. In some embodiments, the oral GVHD is GVHD of the oropharyngeal region. In some embodiments, the oral GVHD is GVHD of the pharyngeal region. In some embodiments, the oral GVHD is GVHD of the esophageal region. In some embodiments, the oral GVHD is acute oral GVHD. In some embodiments, the oral GVHD is chronic oral GVHD. In some embodiments, the subject exhibits one or more symptoms of GVHD.

In some embodiments, the subject exhibits one or more symptoms of GVHD. In some embodiments, the subject exhibits one or more symptoms of acute GVHD. In some embodiments, the subject exhibits one or more symptoms of chronic GVHD.

Exemplary symptoms of GVHD include, but are not limited to, skin rash or reddened areas on the skin, raised skin, blistering, thickening or tightening of the skin, yellow discoloration of the skin and/or eyes, abnormal blood test results, nausea, vomiting, diarrhea, abdominal swelling, abdominal cramping, increased dryness or irritation of the eyes, vision changes, dry mouth, white patches inside the mouth, pain or sensitivity to spicy foods, shortness of breath, difficulty swallowing, pain with swallowing, weight loss, fatigue, muscle weakness, muscle pain, increased urinary frequency, burning or bleeding with urination, vaginal dryness or tightening, or penile dysfunction.

In some embodiments, the subject has or will receive an allogeneic bone marrow or hematopoietic stem cell transplant.

In some embodiments, the subject has or will receive an allogeneic bone marrow transplant.

In some embodiments, the subject has or will receive a hematopoietic stem cell transplant.

In some embodiments, the subject has received an allogeneic bone marrow transplant.

In some embodiments, the subject has received a hematopoietic stem cell transplant.

In some embodiments, the subject will receive an allogeneic bone marrow transplant.

In some embodiments, the subject will receive a hematopoietic stem cell transplant.

In some embodiments, vecabrutinib is administered after the allogeneic bone marrow transplant.

In some embodiments, vecabrutinib is administered after the hematopoietic stem cell transplant.

In some embodiments, vecabrutinib is administered concurrently with the allogeneic bone marrow transplant.

In some embodiments, vecabrutinib is administered concurrently with the hematopoietic stem cell transplant.

In some embodiments, vecabrutinib is administered prior to the allogeneic bone marrow transplant.

In some embodiments, vecabrutinib is administered prior to the hematopoietic stem cell transplant.

In some embodiments, the subject has cancer. In some embodiments, the subject has a hematological malignancy. In some embodiments, the subject has a relapsed or refractory hematological malignancy. In some embodiments, the subject has a B-cell malignancy. In some embodiments, the subject has a T-cell malignancy.

In some embodiments, the subject has a non-Hodgkin's lymphoma. In some embodiments, the subject has a Hodgkin's lymphoma. In some embodiments, the B-cell malignancy is chronic lymphocytic leukemia (CLL), small lymphocytic lymphoma (SLL), diffuse large B-cell lymphoma (DLBCL), follicular lymphoma (FL), activated B-cell diffuse large B-cell lymphoma (ABC-DLBCL), germinal center diffuse large B-cell lymphoma (GCB DLBCL), primary mediastinal B-cell lymphoma (PMBL), Burkitt's lymphoma, immunoblastic large cell lymphoma, precursor B-lymphoblastic lymphoma, mantle cell lymphoma (MCL), B cell prolymphocytic leukemia, lymphoplasmacytic lymphoma, Waldenstrom macroglobulinemia, splenic marginal zone lymphoma, plasma cell myeloma, plasmacytoma, extranodal marginal zone B cell lymphoma, nodal marginal zone B cell lymphoma, mediastinal (thymic) large B cell lymphoma, intravascular large B cell lymphoma, primary effusion lymphoma, or lymphomatoid granulomatosis.

In some embodiments, the subject has a T-cell malignancy. In some embodiments, the T-cell malignancy is peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In some embodiments, the subject has multiple myeloma.

In some embodiments, the subject has a leukemia, a lymphoma, or a myeloma. In some embodiments, the subject has a leukemia. In some embodiments, the subject has a lymphoma. In some embodiments, the subject has a myeloma. In some embodiments, the B-cell malignancy is a non-Hodgkin's lymphoma. In some embodiments, the B-cell malignancy is chronic lymphocytic leukemia (CLL). In some embodiments, the B-cell malignancy is a relapsed or refractory B-cell malignancy. In some embodiments, the B-cell malignancy is a relapsed or refractory non-Hodgkin's lymphoma. In some embodiments, the B-cell malignancy is a relapsed or refractory CLL. In some embodiments, the subject has high risk CLL.

In some embodiments, the subject has a relapsed or refractory hematologic cancer. In some embodiments, the relapsed or refractory hematologic cancer is a leukemia, a lymphoma, or a myeloma. In some embodiments, the relapsed or refractory hematologic cancer is a non-Hodgkin's lymphoma. In some embodiments, the relapsed or refractory hematologic cancer is a Hodgkin's lymphoma. In some embodiments, the relapsed or refractory hematologic cancer is a B-cell malignancy. In some embodiments, the B-cell malignancy is chronic lymphocytic leukemia (CLL), small lymphocytic lymphoma (SLL), diffuse large B-cell lymphoma (DLBCL) follicular lymphoma (FL), activated B-cell diffuse large B-cell lymphoma (ABC-DLBCL), germinal center diffuse large B-cell lymphoma (GCB DLBCL), primary mediastinal B-cell lymphoma (PMBL), Burkitt's lymphoma, immunoblastic large cell lymphoma, precursor B-lymphoblastic lymphoma, mantle cell lymphoma (MCL), B cell prolymphocytic leukemia, lymphoplasmacytic lymphoma, Waldenstrom macroglobulinemia, splenic marginal zone lymphoma, plasma cell myeloma, plasmacytoma, extranodal marginal zone B cell lymphoma, nodal marginal zone B cell lymphoma, mediastinal (thymic) large B cell lymphoma, intravascular large B cell lymphoma, primary effusion lymphoma, or lymphomatoid granulomatosis. In some embodiments, the relapsed or refractory hematologic cancer is a T-cell malignancy. In some embodiments, the T-cell malignancy is peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas. In some embodiments, the subject has a relapsed or refractory multiple myeloma.

In some embodiments, the subject exhibits one or more symptoms of a hematologic cancer. In some embodiments, the subject exhibits one or more symptoms of a B-cell malignancy. In some embodiments, the subject exhibits one or more symptoms of a leukemia, a lymphoma, or a myeloma. In some embodiments, the subject exhibits one or more symptoms such as, but not limited to, abnormal B-cell function, abnormal B-cell size or shape, abnormal B-cell count, fatigue, fever, night sweats, frequent infection, enlarged lymph nodes, paleness, anemia, easy bleeding or bruising, loss of appetite, weight loss, bone or joint pain, headaches, and petechiae.

In some embodiments, the subject has a high risk of cancer recurrence. In some embodiments, the subject is a mammal, such as, but not limited to a human, a non-human primate, mouse, rat, rabbit, goat, dog, cat, or cow. In some embodiments, the mammal is a human. In some embodiments, a high risk of cancer recurrence is determined based on the expression or presence of a biomarker.

In some embodiments, the subject has received one or more prior anticancer agents.

In some embodiments, the anticancer agent is selected from, for example, alemtuzumab, bendamustine, bortezomib, CAL-101, chlorambucil, cyclophosphamide, dexamethasone, docetaxel, doxorubicin, endostatineverolimus, etoposide, fludarabine, fostamatinib, hydroxydaunorubicin, ibritumomab, ifosphamide, lenalidomide, mesalazine, ofatumumab, paclitaxel, pentostatin, prednisone, rituximab, temsirolimus, thalidomide, tositumomab, and vincristine, or a combination thereof.

In some embodiments, vecabrutinib is administered in combination with another prophylactic agent. In some embodiments, vecabrutinib is administered from day 1 to about day 120 following allogeneic bone marrow or hematopoietic stem cell transplant. In some embodiments, vecabrutinib is administered from day 1 to about day 1000 following allogeneic bone marrow or hematopoietic stem cell transplant. In some embodiments, vecabrutinib is administered in combination with one or more additional therapeutic agents.

In some embodiments, the subject has or will receive a donor lymphocyte infusions (DLI). In some embodiments, the subject is administered one or more DLIs. In some embodiments, the subject is administered two or more DLIs. In some embodiments, the DLI comprises CD3+ lymphocytes.

In some embodiments, the subject is administered one or more donor lymphocyte infusions (DLI) following an allogeneic bone marrow or hematopoietic stem cell transplant.

In some embodiments, vecabrutinib is administered concurrently with a DLI following allogeneic bone marrow or hematopoietic stem cell transplant. In some embodiments, vecabrutinib is administered prior to a DLI following an allogeneic bone marrow or hematopoietic stem cell transplant. In some embodiments, vecabrutinib is administered after a DLI following an allogeneic bone marrow or hematopoietic stem cell transplant.

In some embodiments, administering vecabrutinib reduces B-cell activation (e.g., B220+ or CD138+ plasma cells activation) by at least 40%, at least 50%, at least 60%, at least 70%, at least 80%, at least 90% or 100%.

In some embodiments, administering vecabrutinib reduces B-cell proliferation (e.g., B220+ or CD138+ plasma cells proliferation) by at least 40%, at least 50%, at least 60%, at least 70%, at least 80%, at least 90% or 100%.

In some embodiments, administering vecabrutinib reduces plasma B-cells number (e.g., B220+ or CD138+ plasma cells number) in the subject by at least 40%, at least 50%, at least 60%, at least 70%, at least 80%, at least 90% or 100%.

In some embodiments, administering vecabrutinib reduces cytokine secreting B-cells (e.g., IL-10, IL-4/5) in the subject by at least 40%, at least 50%, at least 60%, at least 70%, at least 80%, at least 90% or 100%.

In some such embodiments, administering vecabrutinib inhibits CD4+ T-cell activation and proliferation by at least 40%, at least 50%, at least 60%, at least 70%, at least 80%, at least 90% or 100%.

In some such embodiments, administering vecabrutinib inhibits CD8+ T-cell activation and proliferation by at least 40%, at least 50%, at least 60%, at least 70%, at least 80%, at least 90% or 100%.

In some such embodiments, administering vecabrutinib reduces cytokine secreting CD4+ T-cells by at least 40%, at least 50%, at least 60%, at least 70%, at least 80%, at least 90% or 100%.

In some such embodiments, administering vecabrutinib reduces cytokine secreting CD8+ T-cells by at least 40%, at least 50%, at least 60%, at least 70%, at least 80%, at least 90% or 100%.

In some embodiments, expression of antigen-presentation molecules CD80 and CD86 on B cells were unchanged.

### BTK Inhibitors

In some embodiments, the BTK inhibitor is a small molecule.

In some embodiments, the BTK inhibitor is a reversible inhibitor of BTK.

In some embodiments, the BTK inhibitor inhibits BTK with a half-maximal inhibitor concentration (IC₅₀) of less than about 100 nM, about 90 nM, about 80 nM, about 70 nM, about 60 nM, about 50 nM, about 40 nM, about 30 nM, about 20 nM, about 15 nM, about 10 nM, about 9 nM, about 8 nM, about 7 nM, about 6 nM, about 5 nM, about 4 nM, about 3 nM, about 2 nM, or about 1 nM.

In some embodiments, the BTK inhibitor inhibits BTK with an IC₅₀ of less than about 100 nM.

In some embodiments, the BTK inhibitor inhibits BTK with an IC₅₀ of less than about 90 nM.

In some embodiments, the BTK inhibitor inhibits BTK with an IC₅₀ of less than about 80 nM.

In some embodiments, the BTK inhibitor inhibits BTK with an IC₅₀ of less than about 70 nM.

In some embodiments, the BTK inhibitor inhibits BTK with an IC₅₀ of less than about 60 nM.

In some embodiments, the BTK inhibitor inhibits BTK with an IC₅₀ of less than about 50 nM.

In some embodiments, the BTK inhibitor inhibits BTK with an IC₅₀ of less than about 40 nM.

In some embodiments, the BTK inhibitor inhibits BTK with an IC₅₀ of less than about 30 nM.

In some embodiments, the BTK inhibitor inhibits BTK with an IC₅₀ of less than about 20 nM.

In some embodiments, the BTK inhibitor inhibits BTK with an IC₅₀ of less than about 15 nM.

In some embodiments, the BTK inhibitor inhibits BTK with an IC₅₀ of less than about 10 nM.

In some embodiments, the BTK inhibitor inhibits BTK with an IC₅₀ of less than about 9 nM.

In some embodiments, the BTK inhibitor inhibits BTK with an IC₅₀ of less than about 8 nM.

In some embodiments, the BTK inhibitor inhibits BTK with an IC₅₀ of less than about 7 nM.

In some embodiments, the BTK inhibitor inhibits BTK with an IC₅₀ of less than about 6 nM.

In some embodiments, the BTK inhibitor inhibits BTK with an IC₅₀ of less than about 5 nM.

In some embodiments, the BTK inhibitor inhibits BTK with an IC₅₀ of less than about 4 nM.

In some embodiments, the BTK inhibitor inhibits BTK with an IC₅₀ of less than about 3 nM.

In some embodiments, the BTK inhibitor inhibits BTK with an IC₅₀ of less than about 2 nM.

In some embodiments, the BTK inhibitor inhibits BTK with an IC₅₀ of less than about 1 nM.

In some embodiments, the BTK inhibitor is a reversible inhibitor of a mutant BTK, wherein the mutant BTK comprises a mutation at residue 481, and wherein the mutation is selected from C481R and C481S.

In some embodiments, the BTK inhibitor inhibits wild-type BTK and mutant BTK (e.g., BTKC48TR or BTKC481S), wherein the BTK inhibitor has a half-maximal inhibitor concentration (IC₅₀) for wild-type BTK that is substantially equivalent (e.g., ±1%, ±2%, ±3%, +4%, +5%, +6%, +7%, +8%, +9%, +10%) to its IC₅₀ for mutant BTK.

In some embodiments, the BTK inhibitor inhibits mutant BTK (e.g., BTKC481R or BTKC481S) with a half-maximal inhibitor concentration (IC₅₀) of less than about 100 nM, about 90 nM, about 80 nM, about 70 nM, about 60 nM, about 50 nM, about 40 nM, about 30 nM, about 20 nM, about 15 nM, about 10 nM, about 9 nM, about 8 nM, about 7 nM, about 6 nM, about 5 nM, about 4 nM, about 3 nM, about 2 nM, or about 1 nM.

In some embodiments, the BTK inhibitor inhibits mutant BTK (e.g., BTKC481R or BTKC481S) with an IC₅₀ of less than about 100 nM.

In some embodiments, the BTK inhibitor inhibits mutant BTK (e.g., BTKC481R or BTKC481S) with an IC₅₀ of less than about 90 nM.

In some embodiments, the BTK inhibitor inhibits mutant BTK (e.g., BTKC481R or BTKC481S) with an IC₅₀ of less than about 80 nM.

In some embodiments, the BTK inhibitor inhibits mutant BTK (e.g., BTKC481R or BTKC481S) with an IC₅₀ of less than about 70 nM.

In some embodiments, the BTK inhibitor inhibits mutant BTK (e.g., BTKC481R or BTKC481S) with an IC₅₀ of less than about 60 nM.

In some embodiments, the BTK inhibitor inhibits mutant BTK (e.g., BTKC481R or BTKC481S) with an IC₅₀ of less than about 50 nM.

In some embodiments, the BTK inhibitor inhibits mutant BTK (e.g., BTKC481R or BTKC481S) with an IC₅₀ of less than about 40 nM.

In some embodiments, the BTK inhibitor inhibits mutant BTK (e.g., BTKC481R or BTKC481S) with an IC₅₀ of less than about 30 nM.

In some embodiments, the BTK inhibitor inhibits mutant BTK (e.g., BTKC481R or BTKC481S) with an IC₅₀ of less than about 20 nM.

In some embodiments, the BTK inhibitor inhibits mutant BTK (e.g., BTKC481R or BTKC481S) with an IC₅₀ of less than about 15 nM.

In some embodiments, the BTK inhibitor inhibits mutant BTK (e.g., BTKC481R or BTKC481S) with an IC₅₀ of less than about 10 nM.

In some embodiments, the BTK inhibitor inhibits mutant BTK (e.g., BTKC481R or BTKC481S) with an IC₅₀ of less than about 9 nM.

In some embodiments, the BTK inhibitor inhibits mutant BTK (e.g., BTKC481R or BTKC481S) with an IC₅₀ of less than about 8 nM.

In some embodiments, the BTK inhibitor inhibits mutant BTK (e.g., BTKC481R or BTKC481S) with an IC₅₀ of less than about 7 nM.

In some embodiments, the BTK inhibitor inhibits mutant BTK (e.g., BTKC481R or BTKC481S) with an IC₅₀ f less than about 6 nM.

In some embodiments, the BTK inhibitor inhibits mutant BTK (e.g., BTKC481R or BTKC481S) with an IC₅₀ of less than about 5 nM.

In some embodiments, the BTK inhibitor inhibits mutant BTK (e.g., BTKC481R or BTKC481S) with an IC₅₀ of less than about 4 nM.

In some embodiments, the BTK inhibitor inhibits mutant BTK (e.g., BTKC481R or BTKC481S) with an IC₅₀ of less than about 3 nM.

In some embodiments, the BTK inhibitor inhibits mutant BTK (e.g., BTKC481R or BTKC481S) with an IC₅₀ of less than about 2 nM.

In some embodiments, the BTK inhibitor inhibits mutant BTK (e.g., BTKC481R or BTKC481S) with an IC₅₀ of less than about 1 nM.

In some embodiments, the BTK inhibitor is a reversible inhibitor of BTK and ITK.

In some embodiments, the BTK inhibitor inhibits ITK with a half-maximal inhibitor concentration (IC₅₀) of less than about 100 nM, about 90 nM, about 80 nM, about 70 nM, about 60 nM, about 50 nM, about 40 nM, about 30 nM, about 20 nM, about 15 nM, about 10 nM, about 9 nM, about 8 nM, about 7 nM, about 6 nM, about 5 nM, about 4 nM, about 3 nM, about 2 nM, or about 1 nM.

In some embodiments, the BTK inhibitor inhibits ITK with an IC₅₀ of less than about 100 nM.

In some embodiments, the BTK inhibitor inhibits ITK with an IC₅₀ of less than about 90 nM.

In some embodiments, the BTK inhibitor inhibits ITK with an IC₅₀ of less than about 80 nM.

In some embodiments, the BTK inhibitor inhibits ITK with an IC₅₀ of less than about 70 nM.

In some embodiments, the BTK inhibitor inhibits ITK with an IC₅₀ of less than about 60 nM.

In some embodiments, the BTK inhibitor inhibits ITK with an IC₅₀ of less than about 50 nM.

In some embodiments, the BTK inhibitor inhibits ITK with an IC₅₀ of less than about 40 nM.

In some embodiments, the BTK inhibitor inhibits ITK with an IC₅₀ of less than about 30 nM.

In some embodiments, the BTK inhibitor inhibits ITK with an IC₅₀ of less than about 20 nM.

In some embodiments, the BTK inhibitor inhibits ITK with an IC₅₀ of less than about 15 nM.

In some embodiments, the BTK inhibitor inhibits ITK with an IC₅₀ of less than about 10 nM.

In some embodiments, the BTK inhibitor inhibits ITK with an IC₅₀ of less than about 9 nM.

In some embodiments, the BTK inhibitor inhibits ITK with an IC₅₀ of less than about 8 nM.

In some embodiments, the BTK inhibitor inhibits ITK with an IC₅₀ of less than about 7 nM.

In some embodiments, the BTK inhibitor inhibits ITK with an IC₅₀ of less than about 6 nM.

In some embodiments, the BTK inhibitor inhibits ITK with an IC₅₀ of less than about 5 nM.

In some embodiments, the BTK inhibitor inhibits ITK with an IC₅₀ of less than about 4 nM.

In some embodiments, the BTK inhibitor inhibits ITK with an IC₅₀ of less than about 3 nM.

In some embodiments, the BTK inhibitor inhibits ITK with an IC₅₀ of less than about 2 nM.

In some embodiments, the BTK inhibitor inhibits ITK with an IC₅₀ of less than about 1 nM.

In some embodiments, the BTK inhibitor inhibits BTK and ITK with a half-maximal inhibitor concentration (IC₅₀) that is at least about 10-fold, about 20-fold, about 30-fold, about 40-fold, about 50-fold, about 60-fold, about 70-fold, about 80-fold, about 90-fold, or about 100-fold more potent than its IC₅₀ for one or more TEC family kinase inhibitors selected from TXK, BMK, EGFR, ERBB2, ERBB4, CSK, FGR, BRK, HCK, YES, JAK3, FRK, RET, FLT3, ABL and FYN.

In some embodiments, the BTK inhibitor inhibits BTK and ITK with an IC₅₀ that is at least about 10-fold, about 20-fold, about 30-fold, about 40-fold, about 50-fold, about 60-fold, about 70-fold, about 80-fold, about 90-fold, or about 100-fold more potent than its IC₅₀ for TXK.

In some embodiments, the BTK inhibitor inhibits BTK and ITK with an IC₅₀ that is at least about 10-fold, about 20-fold, about 30-fold, about 40-fold, about 50-fold, about 60-fold, about 70-fold, about 80-fold, about 90-fold, or about 100-fold more potent than its IC₅₀ for BMK.

In some embodiments, the BTK inhibitor inhibits BTK and ITK with an IC₅₀ that is at least about 10-fold, about 20-fold, about 30-fold, about 40-fold, about 50-fold, about 60-fold, about 70-fold, about 80-fold, about 90-fold, or about 100-fold more potent than its IC₅₀ for EGFR.

In some embodiments, the BTK inhibitor inhibits BTK and ITK with an IC₅₀ that is at least about 10-fold, about 20-fold, about 30-fold, about 40-fold, about 50-fold, about 60-fold, about 70-fold, about 80-fold, about 90-fold, or about 100-fold more potent than its IC₅₀ for ERBB2.

In some embodiments, the BTK inhibitor inhibits BTK and ITK with an IC₅₀ that is at least about 10-fold, about 20-fold, about 30-fold, about 40-fold, about 50-fold, about 60-fold, about 70-fold, about 80-fold, about 90-fold, or about 100-fold more potent than its IC₅₀ for ERBB4.

In some embodiments, the BTK inhibitor inhibits BTK and ITK with an IC₅₀ that is at least about 10-fold, about 20-fold, about 30-fold, about 40-fold, about 50-fold, about 60-fold, about 70-fold, about 80-fold, about 90-fold, or about 100-fold more potent than its IC₅₀ for CSK.

In some embodiments, the BTK inhibitor inhibits BTK and ITK with an IC₅₀ that is at least about 10-fold, about 20-fold, about 30-fold, about 40-fold, about 50-fold, about 60-fold, about 70-fold, about 80-fold, about 90-fold, or about 100-fold more potent than its IC₅₀ for FGR.

In some embodiments, the BTK inhibitor inhibits BTK and ITK with an IC₅₀ that is at least about 10-fold, about 20-fold, about 30-fold, about 40-fold, about 50-fold, about 60-fold, about 70-fold, about 80-fold, about 90-fold, or about 100-fold more potent than its IC₅₀ for BRK.

In some embodiments, the BTK inhibitor inhibits BTK and ITK with an IC₅₀ that is at least about 10-fold, about 20-fold, about 30-fold, about 40-fold, about 50-fold, about 60-fold, about 70-fold, about 80-fold, about 90-fold, or about 100-fold more potent than its IC₅₀ for HCK.

In some embodiments, the BTK inhibitor inhibits BTK and ITK with an IC₅₀ that is at least about 10-fold, about 20-fold, about 30-fold, about 40-fold, about 50-fold, about 60-fold, about 70-fold, about 80-fold, about 90-fold, or about 100-fold more potent than its IC₅₀ for YES.

In some embodiments, the BTK inhibitor inhibits BTK and ITK with an IC₅₀ that is at least about 10-fold, about 20-fold, about 30-fold, about 40-fold, about 50-fold, about 60-fold, about 70-fold, about 80-fold, about 90-fold, or about 100-fold more potent than its IC₅₀ for JAK3.

In some embodiments, the BTK inhibitor inhibits BTK and ITK with an IC₅₀ that is at least about 10-fold, about 20-fold, about 30-fold, about 40-fold, about 50-fold, about 60-fold, about 70-fold, about 80-fold, about 90-fold, or about 100-fold more potent than its IC₅₀ for FRK.

In some embodiments, the BTK inhibitor inhibits BTK and ITK with an IC₅₀ that is at least about 10-fold, about 20-fold, about 30-fold, about 40-fold, about 50-fold, about 60-fold, about 70-fold, about 80-fold, about 90-fold, or about 100-fold more potent than its IC₅₀ for RET.

In some embodiments, the BTK inhibitor inhibits BTK and ITK with an IC₅₀ that is at least about 10-fold, about 20-fold, about 30-fold, about 40-fold, about 50-fold, about 60-fold, about 70-fold, about 80-fold, about 90-fold, or about 100-fold more potent than its IC₅₀ for FLT3.

In some embodiments, the BTK inhibitor inhibits BTK and ITK with an IC₅₀ that is at least about 10-fold, about 20-fold, about 30-fold, about 40-fold, about 50-fold, about 60-fold, about 70-fold, about 80-fold, about 90-fold, or about 100-fold more potent than its IC₅₀ for ABL.

In some embodiments, the BTK inhibitor inhibits BTK and ITK with an IC₅₀ that is at least about 10-fold, about 20-fold, about 30-fold, about 40-fold, about 50-fold, about 60-fold, about 70-fold, about 80-fold, about 90-fold, or about 100-fold more potent than its IC₅₀ for FYN.

In some embodiments, the IC₅₀ is measured or determined using an in vitro assay. Assays to assess or quantitate or measure activity of protein tyrosine kinase inhibitors as described are known in the art. Such assays can be conducted in vitro and include assays to assess the ability of an agent to inhibit a specific biological or biochemical function. In some embodiments, kinase activity studies can be performed. Protein tyrosine kinases catalyze the transfer of the terminal phosphate group from adenosine triphosphate (ATP) to the hydroxyl group of a tyrosine residue of the kinase itself or another protein substrate. In some embodiments, kinase activity can be measured by incubating the kinase with the substrate (e.g., inhibitor) in the presence of ATP. In some embodiments, measurement of the phosphorylated substrate by a specific kinase can be assessed by several reporter systems including colorimetric, radioactive, and fluorometric detection. (Johnson, S. A. & T. Hunter (2005) Nat. Methods 2:17.) In some embodiments, inhibitors can be assessed for their affinity for a particular kinase or kinases, such as by using competition ligand binding assays (Ma et al., Expert Opin Drug Discov. 2008 June; 3(6):607-621) From these assays, the half-maximal inhibitory concentration (IC₅₀) can be calculated. IC₅₀ is the concentration that reduces a biological or biochemical response or function by 50% of its maximum. In some cases, such as in kinase activity studies, IC₅₀ is the concentration of the compound that is required to inhibit the target kinase activity by 50%. In some cases, the dissociation constant (K_{d}) and/or the inhibition constant (Kᵢ values) can be determined additionally or alternatively. IC₅₀ and K_{d} can be calculated by any number of means known in the art. The inhibition constant (Kᵢ values) can be calculated from the IC₅₀ and K_{d} values according to the Cheng-Prusoff equation: Kᵢ=ICₛₒ/(l+L/K_{d}), where L is the concentration of the inhibitor (Biochem Pharmacol 22: 3099-3108, 1973). Kᵢ is the concentration of unlabeled inhibitor that would cause occupancy of 50% of the binding sites present in the absence of ligand or other competitors.

PCT patent publication WO2013/185084 (PCT application PCT/US13/44800, filed Jun. 7, 2013), the entirety of which is hereby incorporated herein by reference, describes certain BTK inhibitor compounds. Such compounds include (3R,3′R,4'S)-1′-(6-amino-5-fluoropyrimidin-4-yl)-3-((3-chloro-5-(trifluoromethyl) phenyl)amino)-2-oxo-[1,3′-bipiperidine]-4-carboxamide:

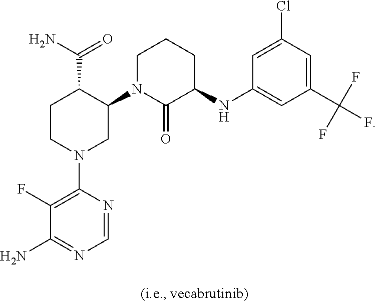 [SMILES: NC(=O)[C@H]1CCN(c2ncnc(N)c2F)C[C@@H]1N1CCC[C@@H](Nc2cc(Cl)cc(C(F)(F)F)c2)C1=O]

In some embodiments, the BTK inhibitor is vecabrutinib, or a pharmaceutically acceptable salt thereof.

In some embodiments, vecabrutinib is provided in a solid form that imparts characteristics such as improved aqueous solubility, stability, absorption, bioavailability, and ease of formulation and isolation.

In some embodiments, the pharmaceutically acceptable salt of vecabrutinib is the succinic acid.

In some embodiments, the succinic acid salt of vecabrutinib depicted as:

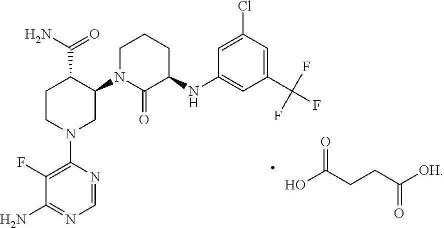

In some embodiments, the succinic acid salt of vecabrutinib has a stoichiometry of (vecabrutinib):(succinic acid) that is about 1:1.

In some embodiments, a pharmaceutical composition of the disclosure comprises vecabrutinib, or a pharmaceutically acceptable salt thereof, and a pharmaceutically acceptable carrier.

In some embodiments, the BTK inhibitor (e.g., vecabrutinib), is administered multiple times in multiple doses. In some embodiments, the BTK inhibitor (e.g., vecabrutinib) is administered once. In some embodiments, the BTK inhibitor (e.g., vecabrutinib), is administered six times daily, five times daily, four times daily, three times daily, twice daily, once daily, every other day, every three days, twice weekly, once weekly, or only one time.

In some embodiments, the dose, frequency, duration, timing and/or order of administration of the BTK inhibitor (e.g., vecabrutinib), is determined, based on particular thresholds or criteria of results of the screening step and/or assessment of treatment outcomes.

In some embodiments, the BTK inhibitor (e.g., vecabrutinib), is administered in a dosage amount of from about 0.2 mg per kg body weight of the subject (mg/kg) to about 200 mg/kg, about 0.2 mg/kg to about 100 mg/kg, about 0.2 mg/kg to about 50 mg/kg, about 0.2 mg/kg to about 10 mg/kg, about 0.2 mg/kg to about 1.0 mg/kg, about 1.0 mg/kg to about 200 mg/kg, about 1.0 mg/kg to about 100 mg/kg, about 1.0 mg/kg to about 50 mg/kg, about 1.0 mg/kg to about 10 mg/kg, about 10 mg/kg to about 200 mg/kg, about 10 mg/kg to about 100 mg/kg, about 10 mg/kg to about 50 mg/kg, about 50 mg/kg to about 200 mg/kg, about 50 mg/kg to about 100 mg/kg, or about 100 mg/kg to about 200 mg/kg. In some embodiments, the BTK inhibitor (e.g., vecabrutinib) is administered at a dose of about 0.2 mg per kg body weight of the subject (mg/kg) to about 50 mg/kg, about 0.2 mg/kg to about 25 mg/kg, about 0.2 mg/kg to about 10 mg/kg, about 0.2 mg/kg to about 5 mg/kg, about 0.2 mg/kg to about 1.0 mg/kg, about 1.0 mg/kg to about 50 mg/kg, about 1.0 mg/kg to about 25 mg/kg, about 1.0 mg/kg to about 10 mg/kg, about 1.0 mg/kg to about 5 mg/kg, about 5 mg/kg to about 50 mg/kg, about 5 mg/kg to about 25 mg/kg, about 5 mg/kg to about 10 mg/kg, or about 10 mg/kg to about 25 mg/kg.

In some embodiments, the BTK inhibitor (e.g., vecabrutinib), is administered in a dosage amount of from about 25 mg to about 2000 mg, about 25 mg to about 1000 mg, about 25 mg to about 500 mg, about 25 mg to about 200 mg, about 25 mg to about 100 mg, about 25 mg to about 50 mg, about 50 mg to about 2000 mg, about 50 mg to about 1000 mg, about 50 mg to about 500 mg, about 50 mg to about 200 mg, about 50 mg to about 100 mg, about 100 mg to about 2000 mg, about 100 mg to about 1000 mg, about 100 mg to about 500 mg, about 100 mg to about 200 mg, about 200 mg to about 2000 mg, about 200 mg to about 1000 mg, about 200 mg to about 500 mg, about 500 mg to about 2000 mg, about 500 mg to about 1000 mg, or about 1000 mg to about 2000 mg.

In some embodiments, the BTK inhibitor (e.g., vecabrutinib), is administered at a total daily dosage amount of at least about 50 mg/day, about 100 mg/day, about 150 mg/day, about 175 mg/day, about 200 mg/day, about 250 mg/day, about 280 mg/day, about 300 mg/day, about 350 mg/day, about 400 mg/day, about 420 mg/day, about 440 mg/day, about 460 mg/day, about 480 mg/day, about 500 mg/day, about 520 mg/day, about 540 mg/day, about 560 mg/day, about 580 mg/day, or about 600 mg/day.

In some embodiments, the BTK inhibitor (e.g., vecabrutinib) is administered at an amount of about 500 mg/day. In some embodiments, the BTK inhibitor (e.g., vecabrutinib) is administered at an amount that is less than about 500 mg/day and at least about 25 mg/day. In some embodiments, the BTK inhibitor (e.g., vecabrutinib) is administered at an amount that is less than about 500 mg/day and at least about 50 mg/day. In some embodiments, the BTK inhibitor (e.g., vecabrutinib) is administered at an amount that is less than about 500 mg/day and at least about 100 mg/day. In some embodiments, the BTK inhibitor (e.g., vecabrutinib) is administered at an amount that is less than about 500 mg/day and at least about 200 mg/day. In some embodiments, the BTK inhibitor (e.g., vecabrutinib) is administered at an amount that is less than 500 mg/day and at least about 300 mg/day.

In some embodiments, the BTK inhibitor (e.g., vecabrutinib) is administered once daily. In some embodiments, the BTK inhibitor (e.g., vecabrutinib) is administered twice daily.

In some embodiments, the BTK inhibitor (e.g., vecabrutinib) is administered orally.

In some embodiments, vecabrutinib is administered in a dosage amount of from about 0.2 mg per kg body weight of the subject (mg/kg) to about 200 mg/kg, about 0.2 mg/kg to about 100 mg/kg, about 0.2 mg/kg to about 50 mg/kg, about 0.2 mg/kg to about 10 mg/kg, about 0.2 mg/kg to about 1.0 mg/kg, about 1.0 mg/kg to about 200 mg/kg, about 1.0 mg/kg to about 100 mg/kg, about 1.0 mg/kg to about 50 mg/kg, about 1.0 mg/kg to about 10 mg/kg, about 10 mg/kg to about 200 mg/kg, about 10 mg/kg to about 100 mg/kg, about 10 mg/kg to about 50 mg/kg, about 50 mg/kg to about 200 mg/kg, about 50 mg/kg to about 100 mg/kg, or about 100 mg/kg to about 200 mg/kg. In some embodiments, vecabrutinib is administered at a dose of about 0.2 mg per kg body weight of the subject (mg/kg) to about 50 mg/kg, about 0.2 mg/kg to about 25 mg/kg, about 0.2 mg/kg to about 10 mg/kg, about 0.2 mg/kg to about 5 mg/kg, about 0.2 mg/kg to about 1.0 mg/kg, about 1.0 mg/kg to about 50 mg/kg, about 1.0 mg/kg to about 25 mg/kg, about 1.0 mg/kg to about 10 mg/kg, about 1.0 mg/kg to about 5 mg/kg, about 5 mg/kg to about 50 mg/kg, about 5 mg/kg to about 25 mg/kg, about 5 mg/kg to about 10 mg/kg, or about 10 mg/kg to about 25 mg/kg.

In some embodiments, vecabrutinib is administered in a dosage amount of from about 25 mg to about 2000 mg, about 25 mg to about 1000 mg, about 25 mg to about 500 mg, about 25 mg to about 200 mg, about 25 mg to about 100 mg, about 25 mg to about 50 mg, about 50 mg to about 2000 mg, about 50 mg to about 1000 mg, about 50 mg to about 500 mg, about 50 mg to about 200 mg, about 50 mg to about 100 mg, about 100 mg to about 2000 mg, about 100 mg to about 1000 mg, about 100 mg to about 500 mg, about 100 mg to about 200 mg, about 200 mg to about 2000 mg, about 200 mg to about 1000 mg, about 200 mg to about 500 mg, about 500 mg to about 2000 mg, about 500 mg to about 1000 mg, or about 1000 mg to about 2000 mg.

In some embodiments, vecabrutinib is administered at a total daily dosage amount of at least about 50 mg/day, about 100 mg/day, about 150 mg/day, about 175 mg/day, about 200 mg/day, about 250 mg/day, about 280 mg/day, about 300 mg/day, about 350 mg/day, about 400 mg/day, about 420 mg/day, about 440 mg/day, about 460 mg/day, about 480 mg/day, about 500 mg/day, about 520 mg/day, about 540 mg/day, about 560 mg/day, about 580 mg/day, or about 600 mg/day.

In some embodiments, vecabrutinib is administered at an amount of about 500 mg/day. In some embodiments, vecabrutinib is administered at an amount that is less than about 500 mg/day and at least about 25 mg/day. In some embodiments, vecabrutinib is administered at an amount that is less than about 500 mg/day and at least about 50 mg/day. In some embodiments, vecabrutinib is administered at an amount that is less than about 500 mg/day and at least about 100 mg/day. In some embodiments, vecabrutinib is administered at an amount that is less than about 500 mg/day and at least about 200 mg/day. In some embodiments, vecabrutinib is administered at an amount that is less than 500 mg/day and at least about 300 mg/day.

In some embodiments, vecabrutinib is administered once daily. In some embodiments, vecabrutinib is administered twice daily.

### Definitions

Unless otherwise stated, the following terms used in the specification and claims have the following meanings set out below.

Without wishing to be limited by this statement, it is understood that, while various options for variables are described herein, the disclosure intends to encompass operable embodiments having combinations of the options. The disclosure may be interpreted as excluding the non-operable embodiments caused by certain combinations of the options.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

It is to be understood that the present disclosure provides methods for the preparation of the combinations described herein.

It is to be understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps order for performing certain actions is immaterial so long as the invention remains operable. Moreover, two or more steps or actions can be conducted simultaneously.

It is to be understood that, unless otherwise stated, any description of a method of treatment or prevention includes use of the combination to provide such treatment or prevention as is described herein. It is to be further understood, unless otherwise stated, any description of a method of treatment or prevention includes use of the combination to prepare a medicament to treat or prevent such condition. The treatment or prevention includes treatment or prevention of human or non-human animals including rodents and other disease models.

It is to be understood that, unless otherwise stated, any description of a method of treatment includes use of the combination to provide such treatment as is described herein. It is to be further understood, unless otherwise stated, any description of a method of treatment includes use of the combination to prepare a medicament to treat such condition. The treatment includes treatment of human or non-human animals including rodents and other disease models.

As used herein, the term “subject” includes human and non-human animals, as well as cell lines, cell cultures, tissues, and organs. In some embodiments, the subject is a mammal. The mammal can be e.g., a human or appropriate non-human mammal, such as primate, mouse, rat, dog, cat, cow, horse, goat, camel, sheep or a pig. The subject can also be a bird or fowl. In some embodiments, the subject is a human.

As used herein, the term “subject in need thereof” refers to a subject having a disease or having an increased risk of developing the disease. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a combination of the present disclosure, or a pharmaceutically acceptable salt, polymorph or solvate of the inhibitor thereof, to alleviate the symptoms or complications of a disease, condition or disorder, or to eliminate the disease, condition or disorder. The term “treat” can also include treatment of a cell in vitro or an animal model. It is to be appreciated that references to “treating” or “treatment” include the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

Treatment of proliferative blood disorders, such as leukemia, lymphoma and myeloma may involve one or more form of chemotherapy and/or radiation therapy. In some embodiments, the treatment of proliferative blood disorders destroy malignant cells, but also destroy healthy blood cells.

Allogeneic hematopoietic cell transplantation is an effective therapy for the treatment of many hematologic malignancies, including, for example, B-cell and T-cell malignancies. In allogeneic hematopoietic cell transplantation, bone marrow (or, in some cases, peripheral blood) from an unrelated or a related donor is used to replace the healthy blood cells destroyed in the cancer patient. The bone marrow (or peripheral blood) contains stem cells, which are the precursors to all the different cell types (e.g., red cells, phagocytes, platelets and lymphocytes) found in blood. Allogeneic hematopoietic cell transplantation is known to have both a restorative effect and a curative effect. The restorative effect arises from the ability of the stem cells to repopulate the cellular components of blood. The curative properties of allogeneic hematopoietic cell transplantation derive largely from a graft-versus-leukemia (GVL) effect. The transplanted hematopoietic cells from the donor (e.g., the T lymphocytes) attack the cancerous cells, enhancing the suppressive effects of the other forms of treatment. Without wishing to be bound by theory, the GVL effect comprises an attack on the cancerous cells by the blood cells derived from the transplantation, making it less likely that the malignancy will return after transplant. Controlling the GVL effect prevents escalation of the GVL effect into GVHD. A similar effect against tumors (graft-versus tumor) is also known.

In some embodiments, the allogeneic hematopoietic cell transplantation is toxic to the subject. In some embodiments, the toxicity arises from the difficulty in dissociating the GVL or GVT effect from graft-versus-host disease (GVHD), an often-lethal complication of allogeneic BMT.

GVHD is a major complication of allogeneic hematopoietic cell transplant (HCT). GVHD is an inflammatory disease initiated by T cells in the donor graft that recognize histocompatibility and other tissue antigens of the host and GVHD is mediated by a variety of effector cells and inflammatory cytokines. GVHD presents in both acute and chronic forms. The most common symptomatic organs are the skin, liver, and gastrointestinal tract, including the oral cavity and oropharyngeal regions. GVHD may involve other organs such as the lung. Treatment of GVHD is generally only 50-75% successful; the remainder of patients generally do not survive. The risk and severity of this immune-mediated condition are directly related to the degree of mismatch between a host and the donor of hematopoietic cells. For example, GVHD develops in up to 30% of recipients of human leukocyte antigen (HLA)-matched sibling marrow, in up to 60% of recipients of FHA-matched unrelated donor marrow, and in a higher percentage of recipient of HLA-mismatched marrow. Patients with mild intestinal GVHD present with anorexia, nausea, vomiting, abdominal pain and diarrhea, whereas patients with severe GVHD are disabled by these symptoms. If untreated, symptoms of intestinal GVHD persist and often progress; spontaneous remissions are unusual. In its most severe form, GVHD leads to necrosis and exfoliation of most of the epithelial cells of the intestinal mucosa, a frequently fatal condition. The symptoms of acute GVHD usually present within 100 days of transplantation. The symptoms of chronic GVHD usually present somewhat later, up to three years after allogeneic HCT, and are often proceeded by a history of acute GVHD.

It is to be understood that a combination of the present disclosure, or a pharmaceutically acceptable salt, polymorph or solvate of the inhibitor thereof, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

It is to be understood that the present disclosure also provides pharmaceutical compositions comprising any combination described herein in combination with at least one pharmaceutically acceptable excipient or carrier.

As used herein, the term “pharmaceutical composition” is a formulation containing the combination of the present disclosure in a form suitable for administration to a subject. In some embodiments, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed peptide or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a peptide of this disclosure include powders, sprays, ointments, pastes, creams, lotions, gels, solutions, patches and inhalants. In some embodiments, the active peptide is mixed under sterile conditions with a pharmaceutically acceptable carrier, and with any preservatives, buffers, or propellants that are required.

As used herein, the term “pharmaceutically acceptable” refers to those combinations, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

It is to be understood that a pharmaceutical composition of the disclosure is formulated to be compatible with its intended route of administration. Examples of routes of administration include parenteral, e.g., intravenous, intradermal, subcutaneous, oral (e.g., ingestion), inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that a combination or pharmaceutical composition of the disclosure can be administered to a subject in many of the well-known methods currently used for chemotherapeutic treatment. For example, a combination of the disclosure may be injected into the blood stream or body cavities. The dose chosen should be sufficient to constitute effective treatment but not so high as to cause unacceptable side effects. The state of the disease condition (e.g., a disease or disorder disclosed herein) and the health of the patient should preferably be closely monitored during and for a reasonable period after treatment.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

As used herein, the term “therapeutically effective amount”, refers to an amount of a pharmaceutical agent to treat or ameliorate an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

It is to be understood that, for any combination, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active combinations of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active peptides into preparations that can be used pharmaceutically. The appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active combination in the required amount in an appropriate solvent with one or a combination of ingredients enumerated above, as required, followed by filtered sterilization. Generally, dispersions are prepared by incorporating the active combination into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, an active component of the combination can be incorporated with excipients and used in the form of tablets, troches, or capsules. Oral compositions can also be prepared using a fluid carrier for use as a mouthwash, wherein the peptide in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or peptides of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active peptides are formulated into ointments, salves, gels, or creams as generally known in the art.

The active combination can be prepared with pharmaceutically acceptable carriers that will protect against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the symptoms of the disease or disorder disclosed herein and also preferably causing complete regression of the disease or disorder. Dosages can range from about 0.01 mg/kg per day to about 5000 mg/kg per day. An effective amount of a pharmaceutical agent is that which provides an objectively identifiable improvement as noted by the clinician or other qualified observer. Improvement in survival and growth indicates regression. As used herein, the term “dosage effective manner” refers to amount of an active peptide to produce the desired biological effect in a subject or cell.

It is to be understood that the pharmaceutical compositions can be included in a container, pack, or dispenser together with instructions for administration.

It is to be understood that, for the inhibitors (e.g., vecabrutinib) of the present disclosure being capable of further forming salts, all of these forms are also contemplated within the scope of the claimed disclosure.

As used herein, the term “pharmaceutically acceptable salts” refer to derivatives of the inhibitors (e.g., vecabrutinib) of the present disclosure wherein the parent inhibitor is modified by making acid or base salts thereof. Examples of pharmaceutically acceptable salts include, but are not limited to, mineral organic acid salts of basic residues such as amines, alkali organic salts of acidic residues such as carboxylic acids, and the like. The pharmaceutically acceptable salts include the conventional non-toxic salts or the quaternary ammonium salts of the parent peptide formed, for example, from non-toxic inorganic organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

In some embodiments, the pharmaceutically acceptable salt is a sodium salt, a potassium salt, a calcium salt, a magnesium salt, a diethylamine salt, a choline salt, a meglumine salt, a benzathine salt, a tromethamine salt, an ammonia salt, an arginine salt, or a lysine salt.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent peptide either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the peptide to the cation or anion of the salt can be 1:1, or any ratio other than 1:1, e.g., 3:1, 2:1, 1:2, or 1:3.

It is to be understood that all references to pharmaceutically acceptable salts include solvent addition forms (solvates) or crystal forms (polymorphs) as defined herein, of the same salt.

The combinations, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally and parenterally. One skilled in the art will recognise the advantages of certain routes of administration.

The dosage regimen utilizing the combination is selected in accordance with a variety of factors including type, species, age, weight, sex and medical condition of the patient; the severity of the condition to be treated; the route of administration; the renal and hepatic function of the patient; and the particular peptide or salt thereof employed. An ordinarily skilled physician or veterinarian can readily determine and prescribe the effective amount of the drug required to prevent, counter, or arrest the progress of the condition. An ordinarily skilled physician or veterinarian can readily determine and prescribe the effective amount of the drug required to counter or arrest the progress of the condition.

Techniques for formulation and administration of the disclosed peptides of the disclosure can be found in Remington: the Science and Practice of Pharmacy, 19^{th} edition, Mack Publishing Co., Easton, PA (1995). In some embodiments, the combinations described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous organic solutions. The combination will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### Biological Assays

Combinations designed, selected and/or optimized by methods described above, once produced, can be characterized using a variety of assays known to those skilled in the art to determine whether the peptides have biological activity. For example, the combination can be characterized by conventional assays, including but not limited to those assays described below, to determine whether they have a predicted activity, binding activity and/or binding specificity.

Various in vitro or in vivo biological assays may be suitable for detecting the effect of the peptides of the present disclosure. These in vitro or in vivo biological assays can include, but are not limited to, enzymatic activity assays, electrophoretic mobility shift assays, reporter gene assays, in vitro cell viability assays, and the assays described herein.

In some embodiments, model of cGVHD (e.g., sclerodermatous cGVHD) may be initiated (e.g., by adoptive transfer of T-cell depleted bone marrow plus whole splenocytes from B10.D2 donors) into a subject (e.g., mouse) that were previously exposes to a form of treatment (e.g., sub-lethal irradiation). The subject may be characterized through a variety of symptoms, including but not limited to weight loss and skin irritation symptoms. The subject may then be treated with a compound of the present disclosure (e.g., vecabrutinib). The subject's body weight and clinical symptoms (e.g., appearance, activity, skin irritation, redness, alopecia and diarrhea) may be measured throughout the study. In some embodiments, the subject's immunophenotyping for B cells and T cells may be performed on organs (e.g., spleens) and measured by a detection method (e.g., flow cytometry). In some embodiments, the subject's levels of circulating immunoglobulins may also be measured (e.g., by a multiplex cytokine assay).

In some embodiments, the biological assay is described in the Examples herein.

### EXEMPLARY EMBODIMENTS

Embodiment 1. In some aspects, the present disclosure provides use of a BTK inhibitor, or a pharmaceutically acceptable salt thereof, for the treatment of chronic graft-versus-host disease (cGVHD).

Embodiment 2. In some aspects, the present disclosure provides a method of treating or preventing chronic graft-versus-host disease (cGVHD), wherein the method comprises administering to a subject a BTK inhibitor, or a pharmaceutically acceptable salt thereof, to a subject in need thereof.

Embodiment 3. In some aspects, the present disclosure provides a BTK inhibitor, or a pharmaceutically acceptable salt thereof, for use in treating or preventing chronic graft-versus-host disease (cGVHD).

Embodiment 4. In some aspects, the present disclosure provides use of a BTK inhibitor, or a pharmaceutically acceptable salt thereof, in the manufacture of a medicament for the treating or preventing chronic graft-versus-host disease (cGVHD).

Embodiment 5. In some aspects, the present disclosure provides the method, use, or inhibitor for use of any one of the preceding Embodiments, wherein treating is reducing the severity of the disease or disorder occurrence in a subject.

Embodiment 6. In some aspects, the present disclosure provides the method, use, or inhibitor for use of any one of the preceding Embodiments, wherein the inhibitor is administered after, prior to, or concurrently with an allogeneic bone marrow or hematopoietic stem cell transplant.

Embodiment 7. In some aspects, the present disclosure provides the method, use, or inhibitor for use of any one of the preceding Embodiments, wherein the cGVHD is liver cGVHD, kidney cGVHD, esophageal cGVHD, stomach cGVHD, sclerodermatous cGVHD, steroid resistant cGVHD, cyclosporin-resistant cGVHD, refractory cGVHD, or oral cGVHD.

Embodiment 8. In some aspects, the present disclosure provides the method, use, or inhibitor for use of any one of the preceding Embodiments, wherein the cGVHD is sclerodermatous cGVHD.

Embodiment 9. In some aspects, the present disclosure provides the method, use, or inhibitor for use of any one of the preceding Embodiments, wherein the subject exhibits one or more symptoms of cGVHD.

Embodiment 10. In some aspects, the present disclosure provides the method, use, or inhibitor for use of Embodiment 9, wherein the subject has cancer.

Embodiment 11. In some aspects, the present disclosure provides the method, use, or inhibitor for use of any one of the preceding Embodiments, wherein the BTK inhibitor is vecabrutinib, or a pharmaceutically acceptable salt thereof.

Embodiment 12. In some aspects, the present disclosure provides the method, use, or inhibitor for use of Embodiment 11, wherein vecabrutinib is provided in a solid form.

Embodiment 13. In some aspects, the present disclosure provides the method, use, or inhibitor for use of Embodiment 11 or Embodiment 12, wherein the pharmaceutically acceptable salt of vecabrutinib is the succinic acid.

Embodiment 14. In some aspects, the present disclosure provides the method, use, or inhibitor for use of any one of Embodiments 11-13, wherein the subject is administered a pharmaceutical composition of vecabrutinib, or a pharmaceutically acceptable salt thereof, and a pharmaceutically acceptable carrier.

Embodiment 15. In some aspects, the present disclosure provides the method, use, or inhibitor for use of any one of the preceding Embodiments, wherein the subject has progressed on one or more prior therapies.

Embodiment 16. In some aspects, the present disclosure provides the method, use, or inhibitor for use of any one of the preceding Embodiments, wherein at least one of the prior therapies was ibrutinib.

Embodiment 17. In some aspects, the present disclosure provides the method, use, or inhibitor for use of any one of the preceding Embodiments, wherein the BTK inhibitor is a reversible inhibitor of a mutant BTK.

Embodiment 18. In some aspects, the present disclosure provides the method, use, or inhibitor for use of Embodiment 17, wherein the mutant BTK comprises a mutation at residue 481.

Embodiment 19. In some aspects, the present disclosure provides the method, use, or inhibitor for use of Embodiment 18, wherein the mutation is selected from C481R and C481S.

### EXAMPLES

### Example 1. Efficacy of Vecabrutinib Treatment in a Murine Model of Sclerodermatous Graft-Versus-Host-Disease

A murine model of sclerodermatous cGVHD was initiated by adoptive transfer of T-cell depleted bone marrow plus whole splenocytes from B10.D2 donors into BALB/c recipients that were previously subjected to sub-lethal irradiation. Total bone marrow and irradiation only groups were included as controls. Mice with established cGVHD characterized by weight loss and skin irritation symptoms were treated with vecabrutinib once daily at 50 mg/kg by oral gavage or ibrutinib at 30 mg/kg in drinking water 5 days per week for approximately 3 weeks beginning on day 27 post-adoptive transfer and ending on day 45 (n=10 mice per group). Body weight and clinical symptoms (appearance, activity, skin symptoms, diarrhea, conjunctivitis) were measured throughout the study. Immunophenotyping for B cells and T cells was performed on spleens collected from euthanized mice by flow cytometry at two timepoints (day 40 during treatment and on day 76 post-treatment). Levels of circulating immunoglobulins were measured by multiplex cytokine assay at both timepoints.

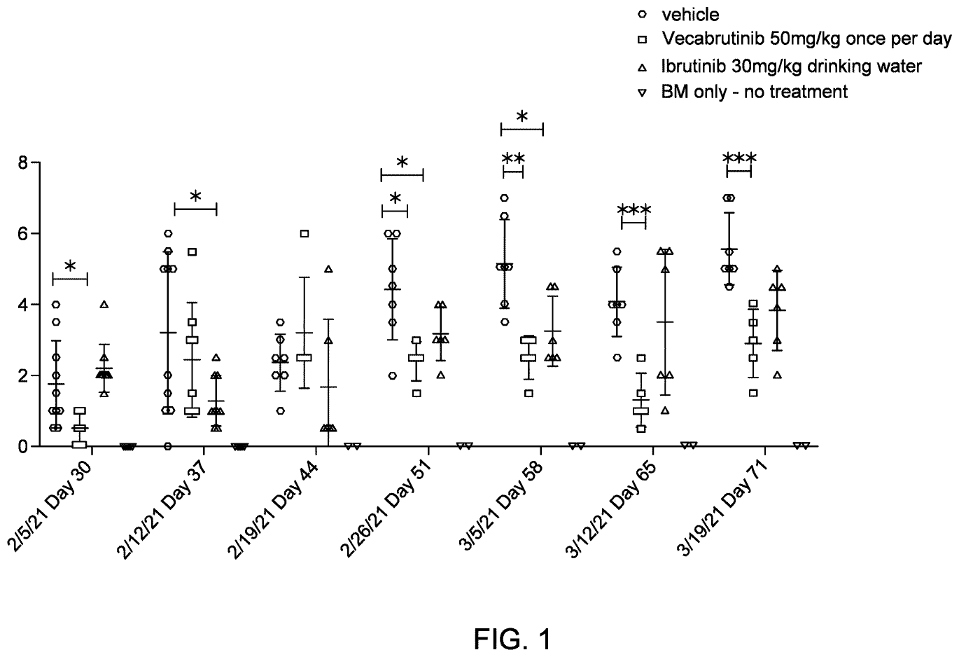

Clinical symptoms, including appearance, activity, skin irritation, redness, alopecia and diarrhea were significantly reduced in vecabrutinib- and ibrutinib-treated groups. Furthermore, there was a trend toward a more favorable clinical score overall for the vecabrutinib-treated group, however no statistical difference was observed compared to ibrutinib possibly due to small sample size. Weight loss was slightly elevated during vecabrutinib and ibrutinib treatment compared to vehicle (trend), however mice recovered body weight post-treatment and continued to maintain benefit. On day 40 (during treatment) and day 76 (post-treatment) groups of mice were euthanized for immunophenotyping analysis utilizing a 22-color flow cytometry panel. Table 1 and FIG. 1 show the average clinical GVHD score per treatment group at three timepoints during the study. Scoring between 0-2 was assigned for each of the parameters (appearance, activity, skin symptoms, diarrhea, conjunctivitis) and totaled for each mouse.

TABLE 1

| | Avg. | | Avg. | | Avg. | |
|---|---|---|---|---|---|---|
| Treatment | Score | Stdev | Score | Stdev | Score | Stdev |
| Group | Day 58 | Day 58 | Day 66 | Day 66 | Day 71 | Day 71 |
| Vehicle | 5.00 | 1.32 | 5.07 | 1.05 | 5.57 | 1.02 |
| (n = 7) | | | | | | |
| Vecabrutinib | 2.50 | 0.61 | 1.30 | 0.75 | 2.90 | 0.96 |
| (n = 5) | | | | | | |
| Ibrutinib | 3.25 | 0.99 | 3.50 | 2.04 | 3.83 | 1.13 |
| (n = 6) | | | | | | |

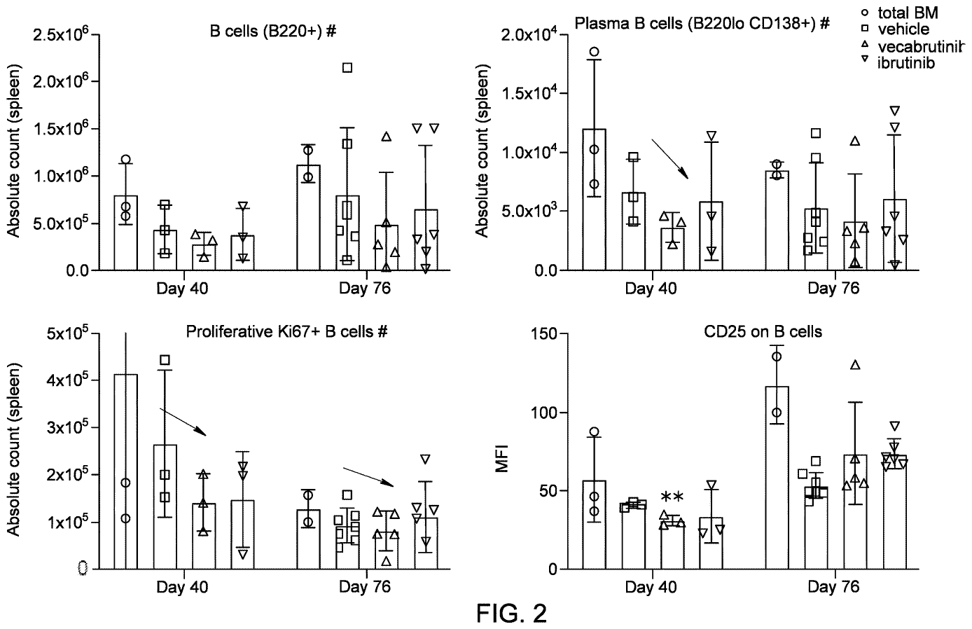

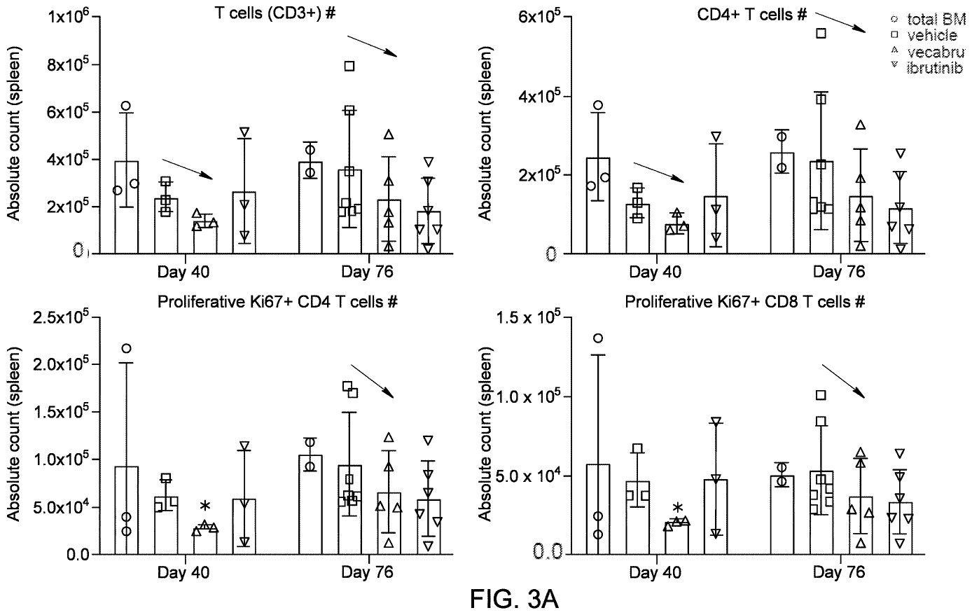

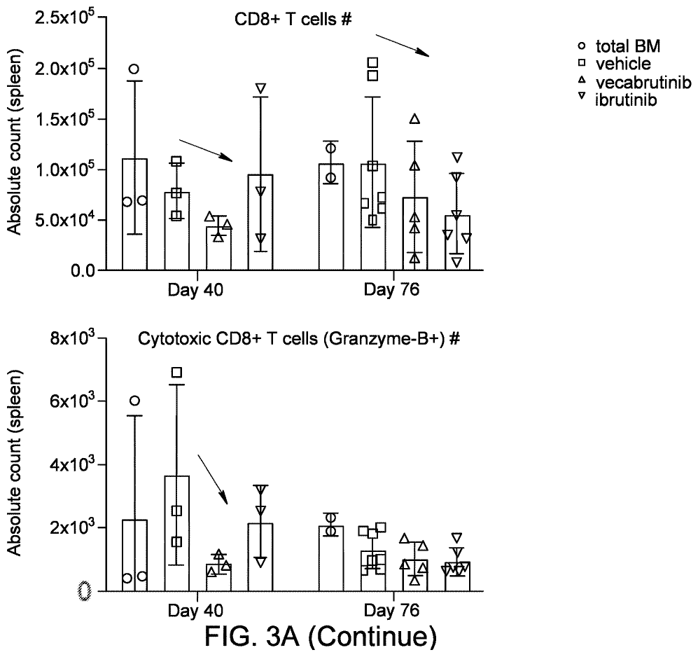

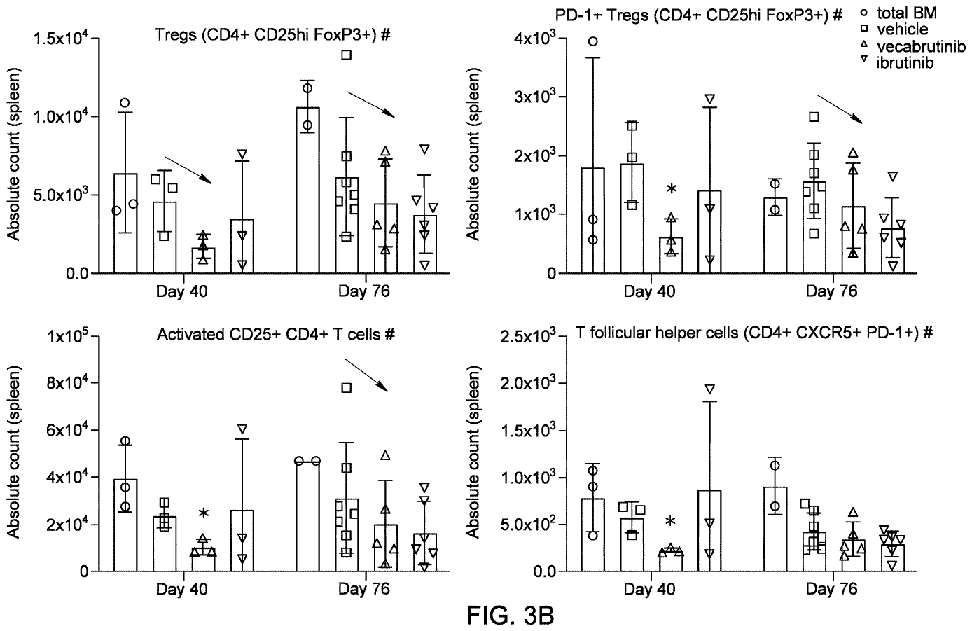

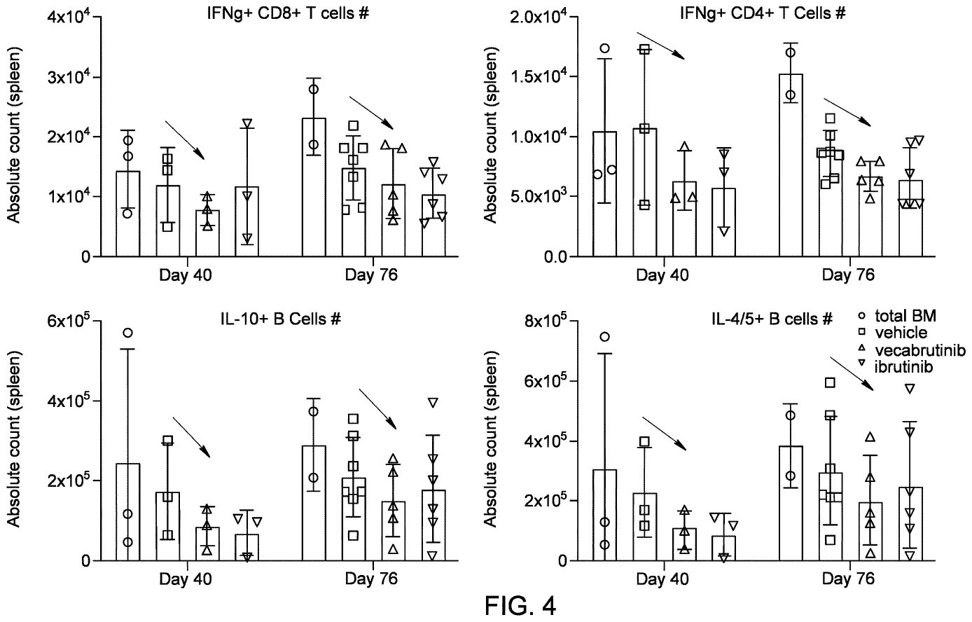

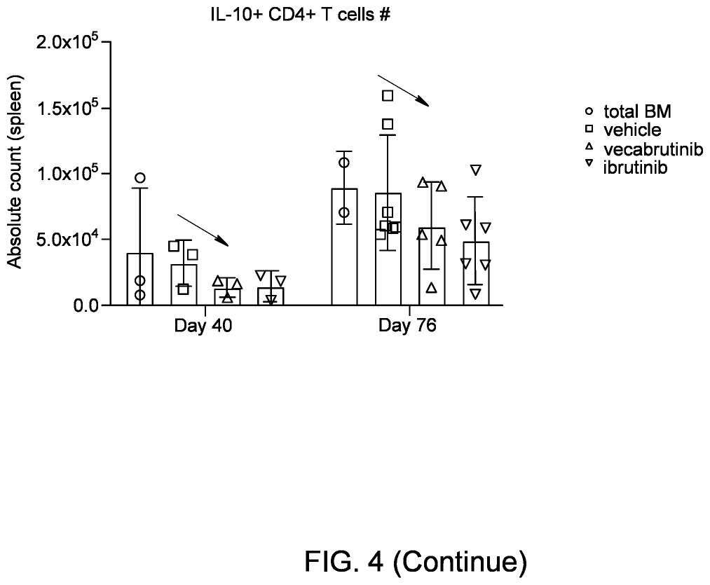

During treatment, both vecabrutinib and ibrutinib-treated mice retained total B cell numbers but exhibited reduced B-cell activation, proliferation, and number of B220+CD138+ plasma cells (FIG. 2). In addition, B cells secreted less IL-10, and IL-4/5 (FIG. 4). Expression of antigen-presentation molecules CD80 and CD86 on B cells were unchanged. Total CD3+ T cells, activated and proliferating CD4+ and CD8+ T cells, and cytotoxic granzyme-B+ CD8+ T cells were reduced in treated mice (FIG. 3A). CD4+CD25+ FoxP3+ Treg number, expression of PD-1 on Tregs and CD4+ CXCR5+PD-1+T follicular helper cells were also reduced in both treatment groups (FIG. 3B). In addition, there were globally reduced numbers of cytokine secreting CD4+ cells but no differences in Th1/Th2 or Th17/Treg ratios were observed.

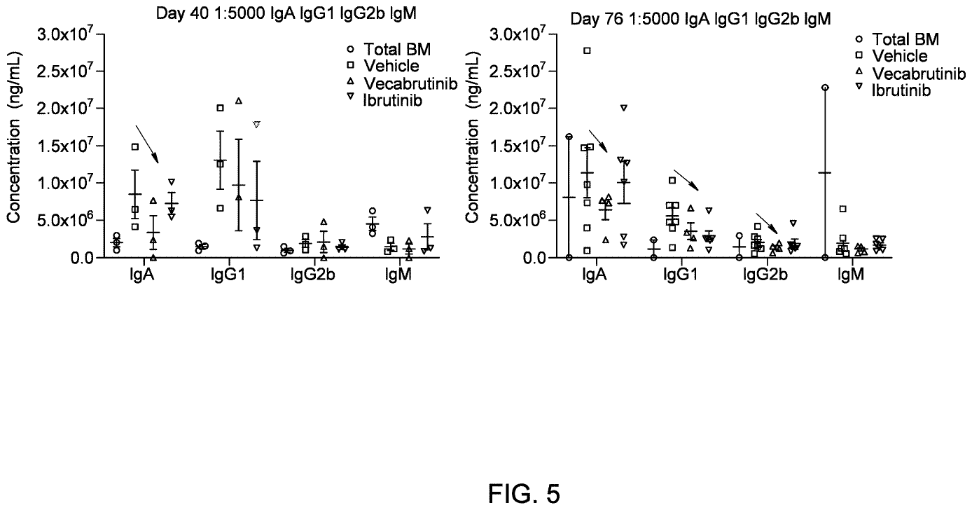

Post-treatment, proliferation and cytokine secretion of B cells and T cells were still lowered but less impaired than during treatment. Tregs and PD-1 expression were still reduced post-treatment. Finally, circulating levels of IgA were reduced during and post treatment in vecabrutinib-treated mice compared to vehicle, while IgG1, IgG2b were reduced in both treated groups (FIG. 5). No changes in IgM levels were observed in either treatment group (FIG. 5). Vecabrutinib treatment demonstrated efficacy and regulated B cell and T cell immune subsets in a preclinical murine model of sclerodermatous cGVHD.

### EQUIVALENTS

The details of one or more embodiments of the invention are set forth in the accompanying description above. Although any methods and materials similar or equivalent to those described herein can be used in the practice or testing of the present invention, the preferred methods and materials are now described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs.

The invention can be embodied in other specific forms without departing from the spirit or essential characteristics thereof. The foregoing description has been presented only for the purposes of illustration and is not intended to limit the invention to the precise form disclosed, but by the claims appended hereto.

1. Use of a BTK inhibitor, or a pharmaceutically acceptable salt thereof, for the treatment of chronic graft-versus-host disease (cGVHD).

2. A method of treating or preventing chronic graft-versus-host disease (cGVHD), wherein the method comprises administering to a subject a BTK inhibitor, or a pharmaceutically acceptable salt thereof, to a subject in need thereof.

3. A BTK inhibitor, or a pharmaceutically acceptable salt thereof, for use in treating or preventing chronic graft-versus-host disease (cGVHD).

4. Use of a BTK inhibitor, or a pharmaceutically acceptable salt thereof, in the manufacture of a medicament for the treating or preventing chronic graft-versus-host disease (cGVHD).

5. The method, use, or inhibitor for use of any one of the preceding claims, wherein treating is reducing the severity of the disease or disorder occurrence in a subject.

6. The method, use, or inhibitor for use of any one of the preceding claims, wherein the inhibitor is administered after, prior to, or concurrently with an allogeneic bone marrow or hematopoietic stem cell transplant.

7. The method, use, or inhibitor for use of any one of the preceding claims, wherein the cGVHD is liver cGVHD, kidney cGVHD, esophageal cGVHD, stomach cGVHD, sclerodermatous cGVHD, steroid resistant cGVHD, cyclosporin-resistant cGVHD, refractory cGVHD, or oral cGVHD.

8. The method, use, or inhibitor for use of any one of the preceding claims, wherein the cGVHD is sclerodermatous cGVHD.

9. The method, use, or inhibitor for use of any one of the preceding claims, wherein the subject exhibits one or more symptoms of cGVHD.

10. The method, use, or inhibitor for use of claim 9, wherein the subject has cancer.

11. The method, use, or inhibitor for use of any one of the preceding claims, wherein the BTK inhibitor is vecabrutinib, or a pharmaceutically acceptable salt thereof.

12. The method, use, or inhibitor for use of claim 11, wherein vecabrutinib is provided in a solid form.

13. The method, use, or inhibitor for use of claim 11 or claim 12, wherein the pharmaceutically acceptable salt of vecabrutinib is the succinic acid.

14. The method, use, or inhibitor for use of any one of claims 11-13, wherein the subject is administered a pharmaceutical composition of vecabrutinib, or a pharmaceutically acceptable salt thereof, and a pharmaceutically acceptable carrier.

15. The method, use, or inhibitor for use of any one of the preceding claims, wherein the subject has progressed on one or more prior therapies.

16. The method, use, or inhibitor for use of any one of the preceding claims, wherein at least one of the prior therapies was ibrutinib.

17. The method, use, or inhibitor for use of any one of the preceding claims, wherein the BTK inhibitor is a reversible inhibitor of a mutant BTK.

18. The method, use, or inhibitor for use of claim 17, wherein the mutant BTK comprises a mutation at residue 481.

19. The method, use, or inhibitor for use of claim 18, wherein the mutation is selected from C481R and C481S.
